(19) United States
(12) Patent Application Publication
(10) Pub. No.: US 2021/0093016 A1
(43) Pub. Date: Apr. 1, 2021

(54) VAPORIZING DEVICE

(71) Applicant: Leonardo GOMEZ, Braintree, MA (US)

(72) Inventor: Leonardo GOMEZ, Braintree, MA (US)

(21) Appl. No.: 16/585,523

(22) Filed: Sep. 27, 2019

(51) Int. Cl. A24F 47/00; A61M 11/04

(52) U.S. Cl. CPC A24F 47/004; A24F 1/02; A61M 11/048

(57) ABSTRACT

A vaporizing device for vaporizing aromatic substances is provided. The vaporizing device includes a heat exchanger configured to heat fresh air to a sub-combustion vaporizing temperature while maintaining the fresh air free from combustion byproducts. The vaporizing device further includes an internal heating cavity configured to permit heating of the aromatic substance via convection.

### FIELD OF THE INVENTION

The present invention is related to a device for vaporizing aromatic substances.

### BACKGROUND

Aromatic substances, including, for example, tobacco, cannabis, and other herbal substances, are frequently vaporized or combusted in recreational use. Conventional devices for such combustion include pipes, bongs, water pipes, and others. Such devices may expose a user to harmful combustion by-products as well as off-putting flavors. Although conventional vaporizers may eliminate the user's exposure to harmful combustion by-products and off-putting flavors, existing vaporizers are intentionally discreet and lack a classic visual appeal. This limits the range of products designed for mature and open consumers of cannabis and other aromatic substances. Furthermore, conventional e-vaporizers have a significant environmental footprint given the range of materials needed to fabricate them and the challenges associated with reclaiming these materials.

Embodiments disclosed herein address the drawbacks of conventional devices in various ways.

### SUMMARY

In an embodiment, a device for heating aromatic substances is provided. The device includes a heating body. The heating body includes an internal heating cavity, at least one internal air flow passage in fluid communication with the internal heating cavity, an aromatic substance support portion disposed in the internal heating cavity, and a heat exchanger coupled to the heating body. The heat exchanger includes a thermal intake and a gas permeable array thermally coupled to the thermal intake and disposed inside the internal heating cavity.

In another embodiment, a device for heating aromatic substances is provided. The device includes a heating body. The heating body includes an internal heating cavity, a lower internal air flow passage configured for coupling with a hollow pipe stem and in fluid communication with the internal heating cavity, an aromatic substance support portion disposed between the internal air flow passage and the internal heating cavity, and an upper internal air flow passage in fluid communication with the internal heating cavity. The device further includes a heat exchanger coupled to the heating body including a thermal intake; and a gas permeable array coupled to the thermal intake such that heat can transfer from the thermal intake to the gas permeable array, the gas permeable array being configured to heat air drawn into the internal heating cavity via the upper internal flow passage in response to a negative pressure at an outlet of the lower internal air flow passage

### BRIEF DESCRIPTION OF THE DRAWINGS

The accompanying figures, which are incorporated herein, form part of the specification and illustrate embodiments of a vaporizing device. Together with the description, the figures further explain the principles of and enable a person skilled in the relevant art(s) to make and use the vaporizing devices described herein. In the drawings, like reference numbers indicate identical or functionally similar elements.

FIG. 1 is a perspective view of a vaporizing device consistent with embodiments hereof.

FIG. 2 is a plan view of a vaporizing device consistent with embodiments hereof.

FIG. 3 is a top view of a heating body consistent with embodiments hereof.

FIG. 4 is a front view of a heating body consistent with embodiments hereof.

FIG. 5 is a side view of a heating body consistent with embodiments hereof.

FIG. 6 illustrates a gas permeable array consistent with embodiments hereof.

FIG. 7 is a perspective view of a heating body consistent with embodiments hereof.

FIG. 8 is a view of upper and lower portions of a heating body consistent with embodiments hereof.

FIG. 9 is a view of upper and lower portions of a heating body consistent with embodiments hereof.

FIG. 10 is an operational flow chart of a method of operating a vaporizing device consistent with embodiments hereof.

### DETAILED DESCRIPTION OF THE INVENTION

Specific embodiments of the present invention are now described with reference to the figures. The following detailed description is merely illustrative in nature and is not intended to limit the invention or the application and uses of the invention. The embodiments discussed below include descriptions of various optional features of vaporizing devices. Although it may be stated that a particular feature or element is included in an embodiment, it is to be understood that, unless explicitly stated otherwise, the features and elements described are optional. The functionality of the various embodiments described herein may be realized without the inclusion of all features and elements that are described. Additionally, the following description is not exhaustive of all combinations of the disclosed features and elements. Even where not explicitly described, all features and elements may be provided in combination with one another unless explicitly stated otherwise.

Embodiments of the devices described herein include vaporization devices configured to heat aromatic substances to sub-combustion temperatures using a heat source applied to a heat exchanger. The heat exchanger permits the aromatic substance to be heated sufficiently to release aromatic vapors from the substance while appropriately throttling the temperature to ensure that combustion by-products from the aromatic substance are minimized. The heat exchanger of embodiments described herein further serves to isolate the fresh air stream that vaporizes the aromatic compounds from the heat source itself, preventing the mixing of combustion by-products from the heat source from being inhaled by a user. The heat exchanger also throttles the temperature of the air stream to sub-combustion vaporization temperatures.

Vaporizing devices consistent with embodiments herein provide sub-combustion vaporization heat through convection. The convective heating of aromatic substances via heated air provides a consistent and even heat throughout the substance, in contrast to devices that utilize conduction for heating. Conductive heating tends to cause uneven heating that can burn portions of the aromatic substance, resulting in off flavors and potentially harmful byproducts.

Vaporizing devices consistent with embodiments herein also provide improved flavor as compared to conventional electric and/or conductive heating devices.

In embodiments, the vaporizing devices described herein are configured to heat and vaporize aromatic substances such that active compounds present in the aromatic substance are released in a gaseous form. Aromatic substances may include any type of herbal or synthetic substance that releases a vapor, smoke, or other gas upon heating. In embodiments, the vapor, smoke, or other gas is inhaled by a user of the vaporizing device for recreational, medicinal, or other purposes. Suitable aromatic substances may include, but are not limited to, cannabis (e.g., marijuana, hashish), tobacco, catnip, mullein, blue lotus, damiana, white sage, and others.

The design of vaporizing devices described herein, also referred to as pipes, are well suited to embodiments that include aesthetically pleasing features, such as stained wood construction. The design of vaporizing devices described herein permits construction from wood and steel, lending the vaporizing devices a traditional eye-pleasing appearance. Vaporizing devices as described herein use an external combustion source and are easy to use. Further, the design of vaporizing devices disclosed herein may include additional features for convenience.

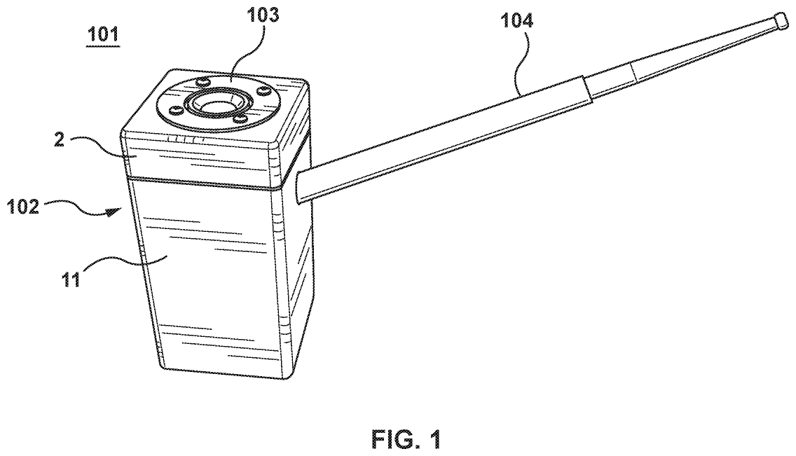

FIG. 1 is a perspective view of a vaporizing device consistent with embodiments hereof. FIG. 1 illustrates a vaporizing device 101 having a heating body 102, a heat exchanger 103, and a stem 104. The heating body 102 includes an upper portion 2 and a lower portion 11.

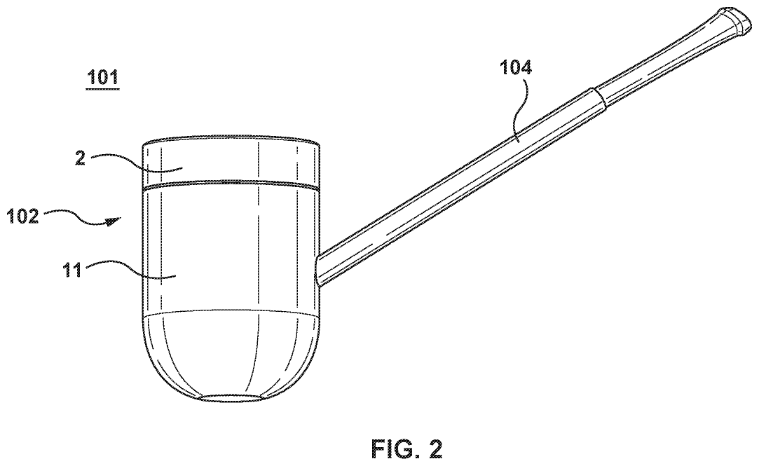

FIG. 2 is a plan view of a vaporizing device consistent with embodiments hereof. FIG. 2 illustrates the vaporizing device 101, the heating body 102, the stem 104, the upper portion 2, and the lower portion 11.

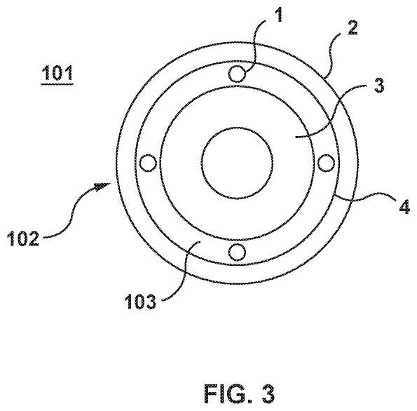

FIG. 3 is a top view of a heating body consistent with embodiments hereof. FIG. 3 illustrates the upper portion 2 of the heating body 102 and external aspects of the heat exchanger 103. Aspects of the heat exchanger 103 visible in FIG. 3 include one or more fasteners 1, clamping ring 4, and thermal intake 3.

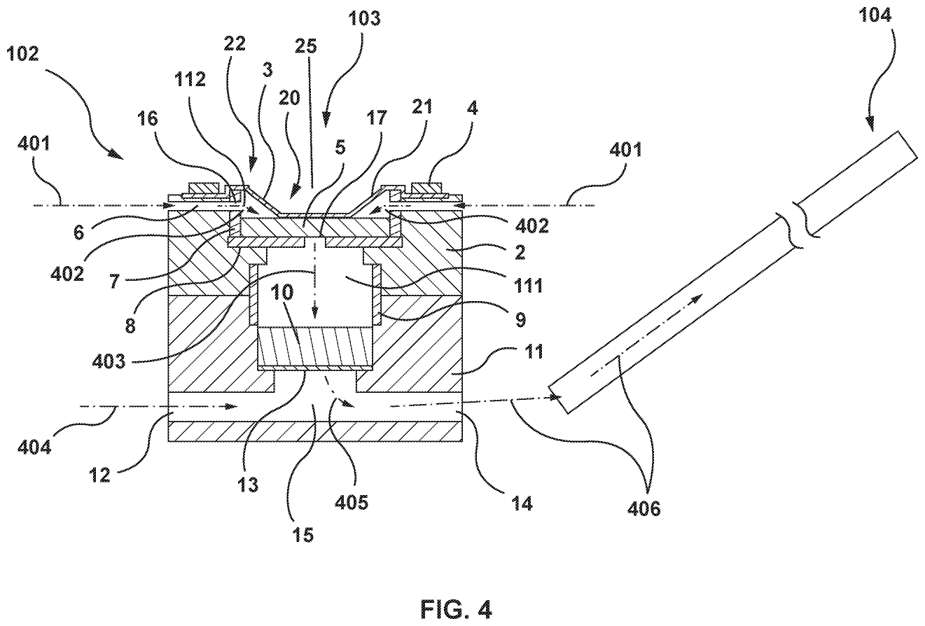

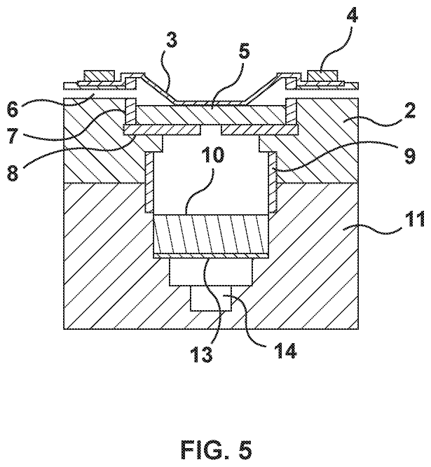

FIG. 4 is a front view of a heating body consistent with embodiments hereof. FIG. 5 illustrates a side view of a heating body consistent with embodiments hereof. FIGS. 4 and 5 illustrate aspects of the heating body 102, the heat exchanger 103, and the stem 104.

The heating body 102 includes the upper portion 2 and lower portion 11. In embodiments, the upper portion 2 and the lower portion 11 are removably coupled by coupling tube 9. The heating body 102 further includes an internal heating cavity 111 formed inside the upper portion 2 and the lower portion 11. In embodiments, the internal heating cavity may be formed partially in the upper portion 2 and partially in the lower portion 11. In further embodiments, the internal heating cavity may be formed completely in one of the upper portion 2 or the lower portion 11.

In embodiments, the heating body 102 further includes a lower internal air flow passage 15 disposed in the lower portion 11 and in fluid communication with the internal heating cavity 111 and having two openings through the heating body 102. The lower internal air flow passage 15 is in fluid communication with the internal heating cavity 111 by any suitably shaped junction, such as a tee junction, wye junction, and/or sanitary tee junction. The junction shape may be selected to appropriately direct air flow. The openings of the lower internal air flow passage 15 are at least one inlet 12 and an outlet 14. The outlet 14 is configured for coupling with the stem 104. The heating body further includes one or more upper internal airflow passages 6 in fluid communication with an upper heating chamber 112. The upper internal airflow passages 6 may include one or more passages, tubes, channels, or conduits that connect the upper heating chamber 112 to the air external to the heating body 102. In an embodiment, the upper internal airflow passages 6 include four passages, one on each side of a square shaped heating body 102. In further embodiments, the upper internal air passages 6 may include any number of passages, e.g., 1, 2, 3, 5, 6, 7, 8, etc. The upper heating chamber 112 is in fluid communication with the internal heating cavity 111. In embodiments, the heating body 102 further includes an aromatic substance support portion 13 configured to contain an aromatic substance 10.

In embodiments, the stem 104 is removably couplable to the heating body 102 at the outlet 14. The stem 104 may be, for example, a plastic or ebonite stem manufactured for tobacco pipes. In such stems, sizing is standardized. Accordingly, outlet 14 may include a hole sized and designed to receive standard tobacco pipe stems. In further embodiments, outlet 14 and stem 104 may be custom-sized and shaped to enhance performance of the vaporizing device 101. In FIG. 4, the stem 104 is illustrated in an uncoupled position with respect to the outlet 14. During operation of the vaporizing device 101, the stem 104 is coupled to the vaporizing device 101 at the outlet 14.

In embodiments, the heat exchanger 103 is coupled to the heating body 102 and includes the thermal intake 3, the clamping ring 4, an exchanger tube segment 7, a gas permeable array 5, and an exchanger support 8. In embodiments, the exchanger tube segment 7 and the exchanger support 8 may be integral. In embodiments, the exchanger support 8 may be omitted. The exchanger tube segment 7 includes one or more holes 16 aligned with the upper internal airflow passage 6 to permit air flow from external to the heating body 102 into the upper heating chamber 112. The gas permeable array 5 is configured to permit fluid/gas flow through it. The exchanger support 8 includes at least one hole 17 permitting gas/fluid flow between the upper heating chamber 112 and the internal heating cavity 111. The at least one hole 17 may include a single hole, a series of holes, a series of perforations, and/or any other hole or opening permitting the passage of fluid/gas.

The structure and operation of the vaporizing device 101 is now described in greater detail with respect to FIGS. 3-5. The thermal intake 3 may be formed of a thin sheet of thermally conductive material, such as metal, e.g., aluminum, copper, brass, steel, etc. In embodiments, the thermal intake 3 has a concave shape, including a bottom intake portion 20, a sloped intake portion 21, and an upper intake portion 22. The bottom intake portion 20 is disposed in the center of the thermal intake 3, the sloped intake portion 21 surrounds the bottom intake portion 20 and the upper intake portion 22 surrounds the sloped intake portion 21. The sloped intake portion 21 is sloped away from the bottom intake portion 20 towards the upper intake portion 22 so as to form the concave or bowl-like shape of the thermal intake 3. In embodiments, the thermal intake 3 is circular, with the bottom intake portion 20 formed as a circle and the sloped intake portion 21 and the upper intake portion 22 formed as progressively larger concentric rings around the bottom intake portion 20. In further embodiments, the thermal intake portion may have alternative shapes, such as a square, hexagon, octagon, star, oval, etc.

The bottom intake portion 20 is in thermal communication with the gas permeable array 5. In embodiments, the bottom intake portion may be directly coupled to the gas permeable array 5. As used herein, “directly coupled” means that that the bottom intake portion 20 contacts the gas permeable array 5 with no intervening materials, parts, or substances. In embodiments, the bottom intake portion may be indirectly coupled to the gas permeable array 5. As user herein, “indirectly coupled” means that a substance, part, or material may intervene between the bottom intake portion 20 and the gas permeable array 5. Any intervening substance, part, or material is selected to maintain thermal communication between the bottom intake portion 20 and the gas permeable array 5. In embodiments, the bottom intake portion 20 may be substantially flat to maintain thermal coupling with the gas permeable array over as large a surface area as possible. In this context, “substantially flat” means that the bottom intake portion 20 is flat within ordinary manufacturing tolerances.

Relative to other vaporizers that require external heat sources the vaporizing device 101 has a very shallow learning curve for operation. The vaporizing device 101 operates by the user first applying heat to the heat exchanger for ˜5-20 seconds. This warms up the gas permeable array 5 within the device, but not the aromatic substance, as the aromatic substance is sufficiently displaced from the heat exchanger so as to prevent early warming/heating. Approximately ˜5-10 seconds prior to removing the heat source the user begins to draw air through the device for the purpose of warming the aromatic substance to vaporization temperatures. The user's inhalation pulls fresh air through the gas permeable array 5 where it is heated and then through the aromatic substance where it vaporizes aromatic compounds present in the substance. The times specified may be adjusted according to adjustments to the structure of the vaporizing device and the heat energy of the external heat source. This process is described in greater detail below.

In operation, a heat source, such as the flame of a butane torch or lighter, is applied to the bottom intake portion 20. The concave shape of the thermal intake 3 serves several purposes. The recess or bowl created by the concave shape serves as a visual marker for the location at which an external heat source is applied. In embodiments, the thermal intake 3 may be marked to further emphasize that it is the target for a heat source. Markings may include, for example, a circle or dot meant as a target, either colored or inscribed, a bullseye or other design, either colored or inscribed, or any other design configured as a visual marker. During operation, heat energy is introduced into the heat exchanger 103 by applying the external heat source directly at and into the concavity of the thermal intake 3. The concavity additionally provides improved efficiency relative to a planar surface due to the larger exposed surface area and the ability to redirect radiation back into the thermal intake 3 for added thermal absorption. In addition, because the external heat is directed into a concavity, the sloping walls of the thermal intake serve to contain any flames and provide additional safety to the user. In embodiments, the thermal intake 3 is constructed from a thin metal sheet to reduce the thermal mass of the thermal intake 3. The reduction in thermal mass ensures that the thermal intake 3 heats quickly to facilitate a more rapid transfer of the thermal energy into the thermal intake 3 and the heat exchanger 103.

In embodiments, the concave shape of the thermal intake 3 also enhances contact between the bottom intake portion 20 and the gas permeable array 5. The clamping ring 4 secures the thermal intake 3 to the upper portion 2 of the heating body 102 via the fasteners 1. The clamping ring 4 presses the thermal intake 2 into the gas permeable array 5. The sloped intake portion 21 of the thermal intake 3 may be formed such that when the thermal intake 3 is unsecured and the bottom intake portion 20 rests on the gas permeable array 5, the upper intake portion 22 does not contact the upper portion 2 of the heating body 102. When the upper intake portion 22 is pressed down and secured to the upper portion 2 of the heating body 102 by the clamping ring 4, the sloped intake portion 21 is caused to flex by force from the gas permeable array 5 on the bottom intake portion 20. Thus, when the thermal intake 3 is properly secured, the bottom intake portion 20 and the gas permeable array 5 exert constant force on one another, ensuring contact. The force exerted on the gas permeable array 5 is transferred to the exchanger support 8, which exerts an opposite force on the gas permeable array 5, ensuring also thermal connection between the gas permeable array 5 and the exchanger support 8.

In embodiments, the clamping ring 4 may also serve as an additional heat sink. The clamping ring 4 may be constructed larger than mechanically necessary so as to provide additional thermal mass. When the thermal intake 3 is heated, the clamping ring 4 is also heated. The additional thermal mass of the clamping ring 4 serves to moderate the temperature of the heat exchanger 103. Thus, if a user keeps the external heat source on the thermal intake 3 for an excessive amount of time, the moderation provided by the clamping ring serves to prevent the heat exchanger from getting hot enough to burn or damage the upper portion 2. The clamping ring 4 may also serve as a convenient surface to rest the external heat source on when heating is applied to the thermal intake 3.

When the thermal intake 3 is heated, energy, in the form of heat, is transferred through the thermal connection between the thermal intake 3 and the gas permeable array 5, causing the gas permeable array 5 to heat. Exchanger tube segment 7 and exchanger support 8 may also be heated through the thermal connection between these elements and the thermal intake.

The heat exchanger 103 is sufficiently displaced from the aromatic substance support portion 13 such that aromatic substances 10 contained by the aromatic substance support portion 13 are substantially not heated when no negative pressure is applied to the outlet 12 of the lower internal air flow passage 15. Thus, after the heat exchanger 103 is brought to temperature by application of the external heat source, the aromatic substance 10 does not begin to heat until the user takes a draw from the stem 104. In this context, substantially not heated means that the temperature of the aromatic substance 10 is not raised enough to induce vaporization. The heat exchanger 103 is disposed such that, when the user takes a draw from the stem 104, applying negative pressure at the outlet 12 of the lower internal air flow passage 15, the aromatic substance 10 is heated to temperatures sufficient for vaporization. Accordingly, during use, the aromatic substance is heated to sub-combustion vaporizing temperatures through convection from the heated air. Such convection provides even and moderate heat. Because conduction does not take a significant role in heating the aromatic substance 10, hot spots and other thermal anomalies caused by contact with conducting surfaces are reduced, preventing the aromatic substance 10 from combusting at the hot spots and producing bad flavors or harmful by products.

After the gas permeable array 5 is heated, the user of the vaporizing device 101 draws air in through the stem 104. Drawing air through the pipe stem creates a negative pressure in at the outlet 14 and therefore in the internal heating cavity 111. The negative pressure at the outlet causes fresh external air to be drawn into the vaporizing device via the upper internal air flow passage 6 and the inlet to the lower internal airflow passage 15.

Due to the negative pressure, fresh air is drawn into the upper heating chamber 112 (arrows 401). The thermal intake 3 secured to the upper portion 2 is, as described above, a solid sheet of material with no holes or perforations. Thus, the thermal intake 2 substantially limits any air flow into the upper heating chamber 112 via the upper opening 25 of the upper portion 2. Substantially all of the fresh air drawn into the upper heating chamber 112 is drawn in through the upper internal air flow passage(s) 6. In this context, “substantially all” means more than 90%, more than 95%, or more than 99% of the fresh air drawn into the upper heating chamber 112. Air in the vicinity of the thermal intake 3 is likely to contain particulates and/or fumes and other combustion byproducts as generated by the heat source used to heat the thermal intake 3. By restricting fresh air intake to the upper internal air flow passage(s) 6, combustion byproducts in the air drawn into the vaporizing device 101 can be reduced, minimized, or eliminated.

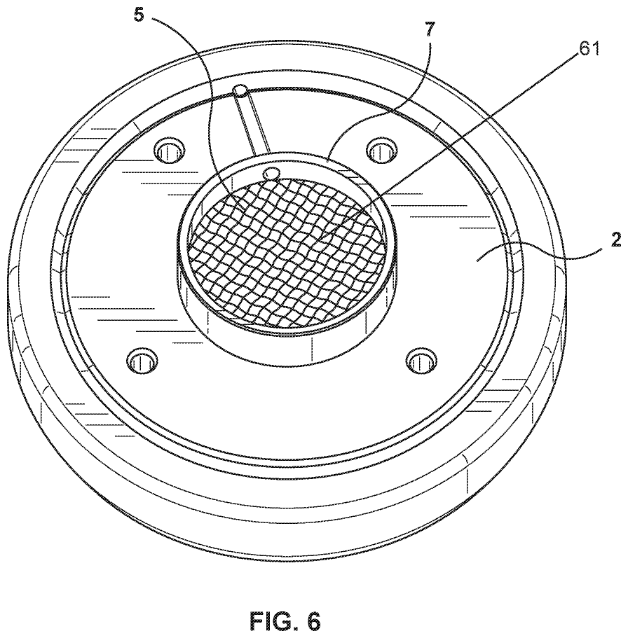

The fresh air (arrows 402) in the upper heating chamber 112 is heated first by contact with the sloped intake portions 21 of the thermal intake 3 before being drawn through the gas permeable array 5 (arrow 403) into the internal heating cavity 111. The fresh air is heated further by contact with portions of the gas permeable array 5, which is described below in greater detail with respect to FIG. 6.

FIG. 6 illustrates a gas permeable array consistent with embodiments hereof The gas permeable array 5 is configured with a high surface area so as to increase the transfer of thermal energy to fresh air flowing through it. In embodiments, the gas permeable array 5 is formed from a plurality of wire mesh layers 61, as illustrated in FIG. 6. In an embodiment, the gas permeable array 5 is constructed of a plurality of wire mesh layers 61, for example, 2, 3, 4, 5, 6, or 7 layers. In further embodiments, the gas permeable array 5 may be any type of structure having a high internal surface area. For example, one or more planar coils of wire, one or more permeable disks, and/or an array of fins may provide a suitable gas permeable array.

Fresh air traveling through the gas permeable array 5 is heated to a sub-combustion chamber relative to common aromatic substances, e.g., between 275-425° F. The gas permeable array 5 is sized and configured such that fresh air passing through it, drawn in at a rate by a negative outlet pressure at a level conventional for a human user to generate, has sufficient thermal mass and sufficient surface area to heat the fresh air to sub-combustion temperatures sufficient for vaporization, e.g., 275-425° F. Sub-combustion temperatures, in this application, are desirable because they reduce, minimize, or eliminate the generation of combustion byproducts as the heated air passes through the aromatic substance 10.

In operation, the temperature of the aromatic substance within the vaporizing device 101 is controlled by variables including the type, nature, and heat output of the heat source used, the dwell time of the heat source on the thermal intake 3, the quantity of aromatic substance in the vaporizing device 101, the flow rate/draw pressure applied by the user, and the duration of the draw. The sub-combustion vaporization temperatures of 275-425° F. may be attained using a butane torch with a dwell time between 5 and 20 seconds, an aromatic substance mass of approximately 0.1-0.5 grams, and a flow/rate and draw pressure applied using a deep, relaxed inhalation. Suitable butane torches may include torches having an adjustable precision flame tip with a maximum temperature of 2700° F. Such a butane torch is an example only, and any suitable external or built-in heat source may be employed. The 5-20 second dwell time is superior to that of electronic vaporizers, which typically require in excess of 30 seconds to one minute of warm up time. Although heating the thermal intake is described herein with respect to use of an external heat source, further embodiments may include heat sources coupled to or internal to the vaporizing device. For example, a gas combustion based heat source, such as a butane torch, may be coupled to or built into the vaporizing device for providing heat to the thermal intake. In further embodiments, resistive heat sources, for example, may be used to directly heat the thermal intake.

In embodiments, the gas permeable array 5 may be sized and configured to meet different requirements. For example, the gas permeable array 5 may be sized and configured to generate heated air at higher or lower temperatures, according to requirements of a preferred aromatic substance. In other embodiments, the gas permeable array 5 may be sized and configured to maintain appropriate temperatures at higher or lower air flow rates and/or higher or lower volume intakes or for longer amounts of time.

As discussed above, the gas permeable array 5 is sized and configured with a large surface area as compared to its overall volume. The large surface area serves to increase heat transfer to the fresh air passing through to appropriate sub-combustion vaporization temperatures. The increase in heat transfer increases the efficiency of the vaporization device 101 as compared with devices that do not include high surface area heat exchangers.

Returning now to FIG. 4, the heated air passes through the aromatic substance 10 (arrow 405) causing vaporization of the aromatic substance 10. After vaporization, a mixture of air and vapors from the aromatic substance 10 flows into the lower internal airflow passage 15. In the lower internal airflow passage 15, the vapor mixture mixes with additional fresh air drawn in via inlet 12 (arrow 404) and then travels out the outlet 14 and up the length of the pipe stem 104 to the user for inhalation (arrows 406). Fresh air entering via inlet 12 may also serve to clear the lower internal airflow passage 15 and stem 104 of vapor. Thus, lower internal airflow passage 15 may be configured to draw fresh air into the stem 104 so as to clear it of vapor after a draw.

The aromatic substance support portion 13 is permeable to fluid/gas flow while maintaining a platform of sufficient solidity and/or rigidity to support the aromatic substance 10. The aromatic substance support portion 13 may, for example, include a perforated disk, a mesh screen, and or any other structure capable of supporting the aromatic substance 10 while permitting fluid/gas flow. The aromatic substance support portion 13 may also serve as or include a particulate filter to prevent particulates from passing into the lower internal airflow passage 15.

The design of the vaporizing device permits the heat exchanger 103 to quickly heat fresh-air to appropriate sub-combustion vaporization temperatures and then to cool off so as to reduce or minimize the possibility of burning a user.

The upper portion 2 and the lower portion 11 are removably coupled to one another. As shown in FIGS. 4 and 5, a coupling tube 9 may facilitate the connection between upper portion 2 and lower portion 11. The upper portion 2 and the lower portion 11 may be held together during use due to a slide or friction fit to the coupling tube 9. In an embodiment, the coupling tube 9 is embedded in the lower portion 11 via a press fit, adhesive, or other means of secure attachment to the lower portion 11. The upper portion 2 may then slide onto the coupling tube 9 to releasably couple the upper portion 2 and the lower portion 11. In further embodiments, the upper portion 2 and the lower portion 11 may be held together during use by magnetic disks, latches, screws, clips, and/or any other suitable fastening mechanism. In further embodiments, the upper portion 2 and the lower portion 11 may be coupled without the use of the coupling tube 9, e.g., by any fastening mechanism as discussed herein.

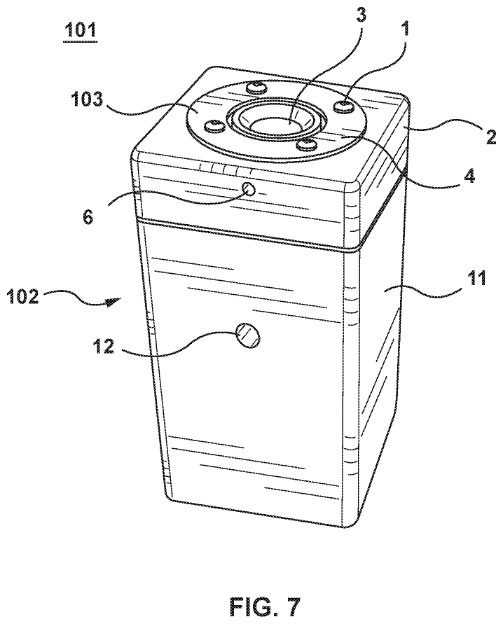

FIG. 7 is a perspective view of a heating body 102 consistent with embodiments hereof. In embodiments, the upper portion 2 and the lower portion 11 are made of hardwood that may be finished with bees wax and orange oil. The bees wax serves to seal the wood to facilitate air flow in the device through functional air channels and prevent air or vapor from seeping into or permeating other portions of the device. The orange oil provides a compensatory scent relative to the aromatic substance 10 in the vaporizing device 101 when not in use. Sealants other than bees wax, such as hemp, linseed, or tung oil may be used in addition to other comparable sealing waxes and oils may be used. Scents other than orange oil, such as any combination of essential oils or scents (e.g. lavender, hemp, tea tree, lemon, sandal wood, rosemary) may be used. Although illustrated as a rectangular prism, the heating body may be formed in any suitable shape, including cylinders, triangular prisms, and others.

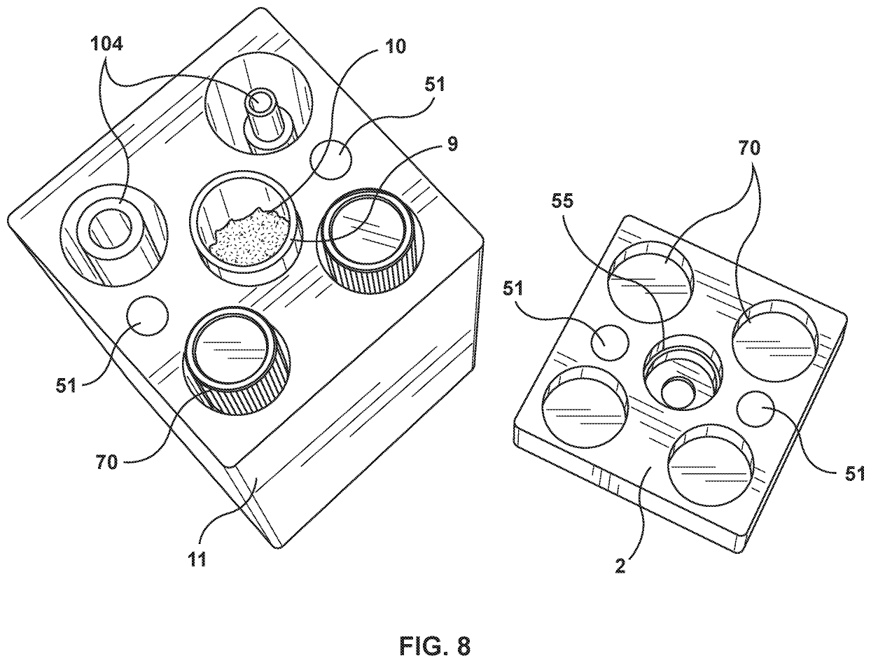

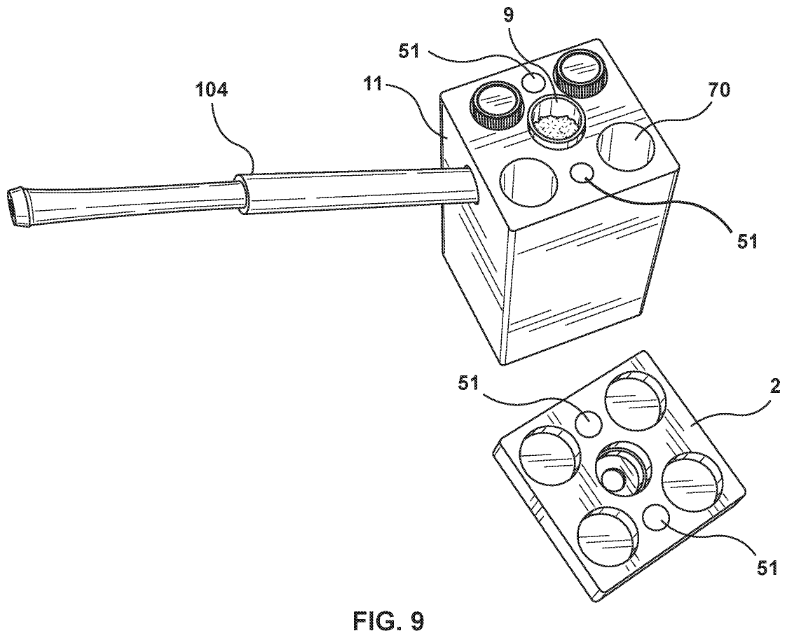

FIGS. 8 and 9 are views of the upper and lower portions of a heating body consistent with embodiments hereof. As shown, the upper portion 2 is separated from the lower portion 11, revealing details of the internal construction. Coupling tube 9 is embedded into the lower portion 11 and extends upwards to releasably couple with the recess 55 in the upper portion 2. The upper portion 2 and the lower portion 11 may each include one or more magnets 51 configured to secure these portions to one another when coupled. In embodiments, magnets 51 may be included in either the upper portion 2 or the lower portion 11 while the portion without the magnets 51 includes a material to which the magnets are attracted.

The lower portion 11 includes one or more storage recesses 70. The upper portion 2 may also include storage recesses 70 which align with the storage recesses 70 of the lower portion 11. The storage recesses 70 are accessible when the lower portion 11 is separated from the upper portion 2. The storage recesses 70 may be used to store accessories for the vaporizing device 101, including a stem 104 (shown in FIG. 7 separated into two parts—a mouthpiece and tube portion), a heat source, an aromatic substance 10, etc.

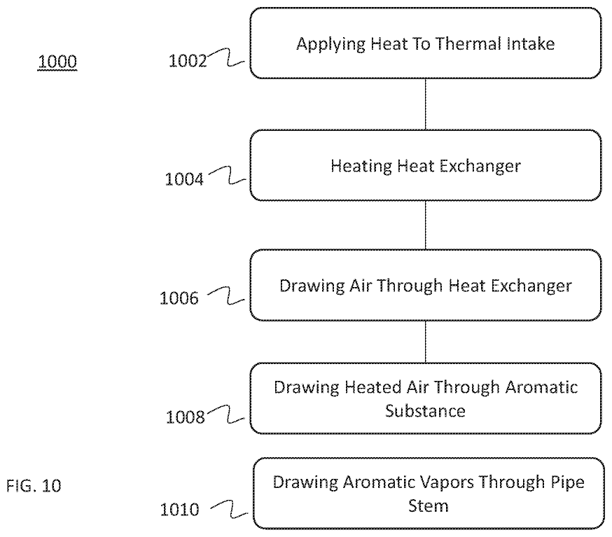

FIG. 10 illustrates a process 1000 of operating a vaporizing device consistent with embodiments hereof. The process 1000 may be employed in the operation of any vaporizing device embodiments disclosed herein, including vaporizing device 101 and all embodiments thereof.

In an operation 1002, process 1000 includes applying heat to a thermal intake of a heat exchanger of the vaporizing device. Heat is applied to a thermal intake through the use of a heat source as described herein. Suitable heat sources may include, for example, a butane torch.

In an operation 1004, process 1000 includes heating a heat exchanger of the vaporizing device. When heat is applied to the thermal intake, all components of the heat exchanger, including a gas permeable array, are heated due to thermal coupling between each component and the thermal intake.

In an operation 1006, process 1000 includes drawing air through the heat exchanger. Negative outlet pressure may be applied to an outlet of a lower internal air flow passage of the vaporizing device. The negative outlet pressure causes air to be drawn into the vaporizing device through an upper internal air flow passage and through the heat exchanger into an internal heating cavity of the vaporizing device. When the air passes through the gas permeable array of the heat exchanger, it is heated to sub-combustion vaporization temperatures.

In an operation 1008, process 1000 includes drawing the heated air through an aromatic substance disposed within the internal heating cavity of the device. The heated air, at sub-combustion vaporization temperatures, causes vaporization of the aromatic substance without causing combustion. After passing through the aromatic substance, the heated air, now mixed with aromatic vapors emitted by the aromatic substance, enters a lower internal air flow passage of the vaporizing device.

In an operation 1010, process 1000 includes drawing the aromatic vapors through a pipe stem of the vaporizing device for inhalation.

Accordingly, the process 1000 makes use of the vaporizing devices, as described herein, to vaporize aromatic substances for inhalation by a user.

The foregoing description has been presented for purposes of illustration and enablement and is not intended to be exhaustive or to limit the invention to the precise form disclosed. Other modifications and variations are possible in light of the above teachings. The embodiments and examples were chosen and described in order to best explain the principles of the invention and its practical application and to thereby enable others skilled in the art to best utilize the invention in various embodiments and various modifications as are suited to the particular use contemplated. It is intended that the appended claims be construed to include other alternative embodiments of the invention.

What is claimed is:

1. A device for heating aromatic substances comprising:
a heating body including:
an internal heating cavity,
a lower internal air flow passage configured for coupling with a hollow pipe stem and in fluid communication with the internal heating cavity,
an aromatic substance support portion disposed between the internal air flow passage and the internal heating cavity, and
an upper internal air flow passage in fluid communication with the internal heating cavity; and
a heat exchanger coupled to the heating body including:
a thermal intake configured to receive heat energy, and
a gas permeable array coupled to the thermal intake such that heat can transfer from the thermal intake to the gas permeable array, the gas permeable array being configured to heat air drawn into the internal heating cavity via the upper internal flow passage in response to a negative pressure at an outlet of the lower internal air flow passage to generate a heated air stream.

2. The device of claim 1, wherein the heating body further includes:
a lower portion, and
an upper portion separable from the lower portion.

3. The device of claim 2, wherein the heating body includes at least one of:
a coupling tube configured to couple the lower portion to the upper portion, and at least one magnet configured to couple the lower portion to the upper portion.

4. The device of claim 3, wherein the outlet of the lower internal air flow passage is included at a first side of the lower portion to which a pipe stem is couplable and wherein the lower internal air flow passage further includes an inlet at a second side of the lower portion.

5. The device of claim 1, wherein the internal air flow passage is in fluid communication with the internal heating cavity at a T-junction.

6. The device of claim 1, wherein the aromatic substance support portion is configured to contain an aromatic substance that is caused to vaporize by the heated air stream.

7. The device of claim 1, wherein the lower internal air passage is configured for clearing a coupled pipe stem of vapor.

8. The device of claim 1, wherein the aromatic substance support portion includes a particulate filter.

9. The device of claim 1, wherein the thermal intake is secured to the heating body via a clamping ring that presses the thermal intake to the gas permeable array.

10. The device of claim 9, wherein the thermal intake is concave, having a lower portion in contact with the gas permeable array, a sloping portion, and an upper portion secured to the heating body.

11. The device of claim 10, wherein the thermal intake is configured such that the sloping portion flexes when the clamping ring presses the lower portion to the gas permeable array.

12. The device of claim 1, wherein the gas permeable array is formed from a plurality of wire mesh layers.

13. The device of claim 1, wherein the thermal intake is secured to an upper opening of the internal heating cavity and is configured to substantially limit air flow into the upper opening of internal heating cavity.

14. The device of claim 1, wherein the thermal intake is thermally coupled to the gas permeable array.

15. The device of claim 1, wherein the heat exchanger is configured to maintain sub-combustion temperatures relative to the aromatic substance within the heated air stream.

16. The device of claim 1, wherein the heat exchanger is displaced from the aromatic substance support portion such that aromatic substances contained by the aromatic substance support portion are substantially not heated when the thermal intake receives heat energy and negative pressure is not applied to the outlet of the lower internal air flow passage.

17. The device of claim 1, wherein the heat exchanger is displaced from the aromatic substance support portion such that aromatic substances contained by the aromatic substance support portion are heated when the thermal intake receives heat energy and negative pressure is applied to the outlet of the lower internal air flow passage.

18. The device of claim 1, wherein the heating body includes one or more storage recesses.

19. The device of claim 18, wherein the heating body further includes:
a lower portion, and
an upper portion separable from the lower portion, and
wherein the one or more storage resources are disposed to be accessible when the lower portion is separated from the upper portion.

20. A device for heating aromatic substances comprising:
a heating body including:
an internal heating cavity,
at least one internal air flow passage in fluid communication with the internal heating cavity,
an aromatic substance support portion disposed in the internal heating cavity, and
a heat exchanger coupled to the heating body including:
a thermal intake;
a gas permeable array thermally coupled to the thermal intake and disposed inside the internal heating cavity.
